(19) United States
(12) Patent Application Publication
(10) Pub. No.: US 2013/0089931 A1
(43) Pub. Date: Apr. 11, 2013

(54) MICRODEVICE FOR FUSING CELLS

(71) Applicants: Sang-Woo JOO, Gyeongsan-si (KR); Shizhi Qian, Gyeongsan-si (KR); Ning Hu, Gyeongsan-si (KR)

(72) Inventors: Sang-Woo JOO, Gyeongsan-si (KR); Shizhi Qian, Gyeongsan-si (KR); Ning Hu, Gyeongsan-si (KR)

(21) Appl. No.: 13/282,874

(22) Filed: Oct. 27, 2011

(30) Foreign Application Priority Data
Oct. 6, 2011 (KR) 10-2011-0101884

(51) Int. Cl. C12N 15/02; C12M 1/42

(52) U.S. Cl. 435/450; 435/285.2

(57) ABSTRACT

A microdevice for fusing cells including: a membrane with a plurality of pores having a diameter smaller than the smallest diameter among the first kind of cells and second kind of cells; a first chamber where the first cell is located and a second chamber where the second cell is located, wherein the membrane is disposed therebetween; a first electrode combined to the first chamber; a second electrode combined to the second chamber; and a power generator applying a voltage to the first and second electrodes. Accordingly, the first and second cells across the membrane may be arranged in a one-to-one manner between the first and second electrodes, and thus the first and second cells having different traits may be smoothly fused in a one-to-one manner when electric signals are sequentially applied thereto.

### CROSS-REFERENCE TO RELATED PATENT APPLICATION

This application claims the benefit of Korean Patent Application No. 10-2011-0101884, filed on Oct. 6, 2011, in the Korean Intellectual Property Office, the disclosure of which is incorporated herein in its entirety by reference.

### BACKGROUND OF THE INVENTION

1. Field of the Invention

The present invention relates to a microdevice for cell electrofusion, which manufactures a desired fused cell at high efficiency.

2. Description of the Related Art

Cell fusion is a method of preparing a hybrid cell by artificially fusing two cells in different types. The cell fusion may be performed by using chemicals or an electric pulse. Here, combining two cells in different types by porating a cell membrane via an electric pulse is referred to as electrofusion.

There are mainly four continuous phases in the electrofusion: dielectrophoresis-based cell alignment, reversible electroporation, reconstruction of cytomembrane, and karyon fusion. Generally, the dielectrophoresis-based cell alignment needs a sinusoidal alternating current (AC) electric field (intensity: 100 to 300 V/cm) to exert a positive dielectrophoretic (DEP) force on the cells. In addition, a high-strength DC electric pulse signal series is required in the reversible electroporation (intensity: 1 to 10 kV/cm, pulse width: 10 to 50 μs).

Plate electrodes are usually used in a conventional cell electrofusion device. In general, a distance between two plate electrodes is equal to or above 1 cm, and as a result, an expensive generator is required to obtain high-strength electric pluses. Moreover, an electric field generated between the plate electrodes is uniform, and thus probabilities of occurrence of reversible electroporation and electrofusion of aligned cells are equal. Thus, a probability of occurrence of unwanted multi-cell electrofusion in the conventional cell electrofusion device is relatively high.

In order to increase pairing precision, fusion efficiency, multi-function integration, and a degree of automation, a micro electromechanical system (MEMS) and microfluidic technology have been used to develop microchips for electrofusion. Microstructures in these microchips have a similar scale as cells (5 to 50 μm), and thus useful in more precise cell manipulation. Also, owing to a short distance between two microelectrodes, a high electric field required for cell fusion may be generated even with a low voltage, and thus difficulties of power supply and high manufacturing costs may be reduced.

However, in a conventional microfluidic device, an average cell fusion efficiency is about 40%, which is higher than a general chemical fusing method (use polyethylene glycol (PEG), less than 5%) and a conventional electrofusion method (less than or equal to 12%), but a probability of forming desired cell-cell twins is only from 42 to 68%. Accordingly, fusion efficiency of total cells is about 40%×42-68%, i.e., 16 to 30%. In other words, when a cell A and a cell B are to be fused, undesired hybrid products, such as AA, ABB, AABB, AAB, and BB, may be excessively obtained instead of AB.

Accordingly, a new microfluidic chip for fusing desired cells at higher efficiency is required to be developed.

### SUMMARY OF THE INVENTION

The present invention provides a microdevice for fusing cells, wherein cells to be fused are effectively fused in a one-to-one manner.

According to an aspect of the present invention, there is provided a microdevice for fusing cells including: a membrane with a plurality of pores having a diameter smaller than the smallest diameter among the first and second kinds of cells; a first chamber where the first cell is located and a second chamber where the second cell is located, wherein the membrane is disposed therebetween; a first electrode combined to the first chamber; a second electrode combined to the second chamber; and a power generator applying cell electrofusion signals to the first and second electrodes.

According to another aspect of the present invention, there is provided a method of fusing cells, the method including: providing the microdevice; injecting the first cell into the first chamber and injecting the second cell into the second chamber; applying an alternating current (AC) voltage between the first and second electrodes such that the first and second cells are disposed across the pore of the membrane according to a positive dielectrophoresis; performing electroporation by applying direct current (DC) pulses between the first and second electrodes; applying a quasi-damping AC voltage between the first and second electrodes such that the electroporated first and second cells are close contacted and fused by being adjacently disposed to each other according to a positive dielectrophoresis; and obtaining the fused hybrid cells.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view of a microdevice for fusing cells, according to an embodiment of the present invention;

FIG. 2 is an exploded perspective view of the microdevice, according to an embodiment of the present invention;

FIG. 3 is a perspective view of a membrane according to an embodiment of the present invention;

FIG. 4 is a perspective view of a chamber according to an embodiment of the present invention;

FIG. 5 is a perspective view of an upper cover according to an embodiment of the present invention; and

FIGS. 6A through 6D are schematic internal cross-sectional views for describing operations of a microdevice for fusing cells.

### DETAILED DESCRIPTION OF THE INVENTION

Hereinafter, embodiments of the present invention will be described in detail. The invention may, however, be embodied in many different forms and should not be construed as being limited to the embodiments set forth herein; rather, these embodiments are provided so that this disclosure will be thorough and complete, and will fully convey the concept of the invention to those skilled in the art.

The present invention will be described more fully with reference to the accompanying drawings.

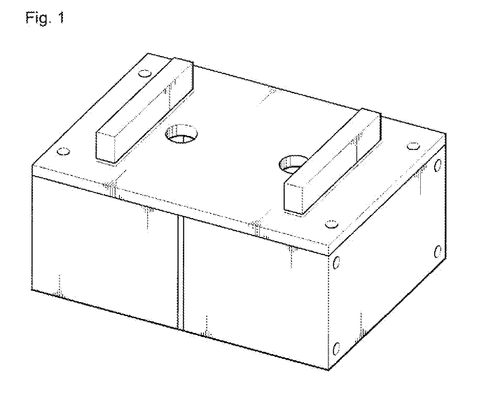

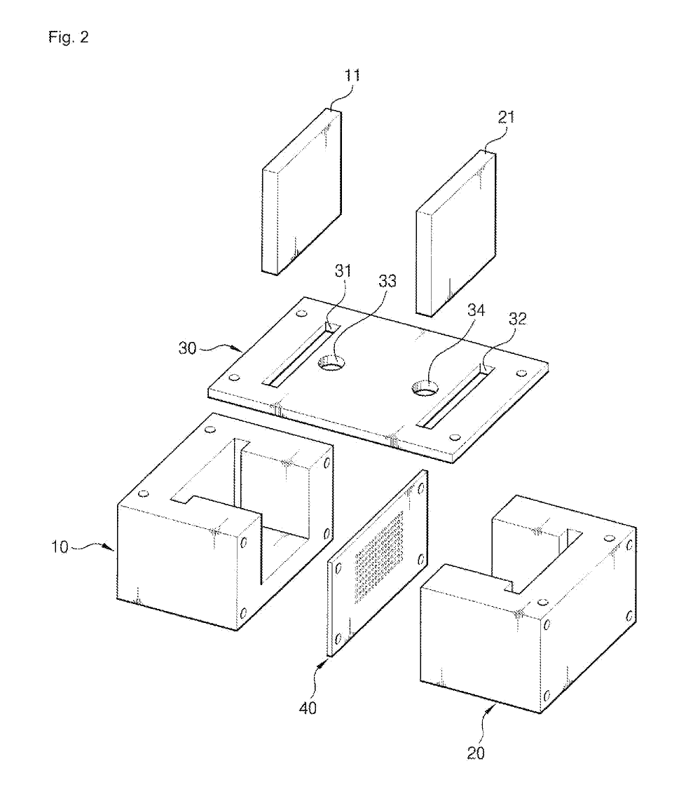

FIG. 1 is a perspective view of a microdevice for fusing cells, according to an embodiment of the present invention, and FIG. 2 is an exploded perspective view of the microdevice. For convenience of illustration, a power generator connected between first and second electrodes is not shown, but the power generator would have been obvious to one of ordinary skill in the art.

An embodiment of the present invention provides a microdevice for fusing cells including: a membrane with a plurality of pores having a diameter smaller than the smallest diameter among the first and second kinds of cells; a first chamber 10 where the first cell is located and a second chamber 20 where the second cell is located, wherein the membrane is disposed therebetween; a first electrode 11 combined to the first chamber 10; a second electrode 21 combined to the second chamber 20; and a power generator applying a voltage to the first and second electrodes.

According to the current embodiment of the present invention, a membrane 40 includes a plurality of pores 41, and divides a first chamber 10 and a second chamber 20. The first chamber 10 and the second chamber 20 are divided by the membrane 40 to have individual spaces.

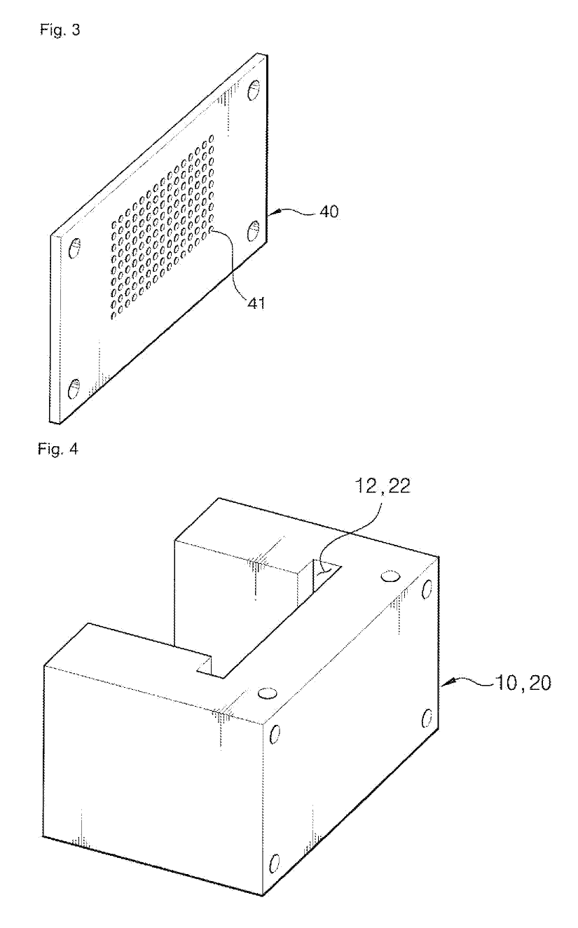

FIG. 3 is a perspective view of the membrane 40 according to an embodiment of the present invention.

According to an embodiment of the present invention, the pores 41 included in the membrane 40 may be arranged in a lattice shape, but are not limited thereto. Also, diameters of the pores 41 depend on diameters of first and second cells existing respectively in the first and second chambers 10 and 20. The diameters of the pores 41 are formed to be smaller than the smallest diameter among the first and second cells respectively existing in the first and second chambers 10 and 20 so as to prevent the first and second cells from freely moving. In detail, the diameters of the pores 41 may be smaller by 1 to 15 μm than the smaller diameter from among the first and second cells, but are not limited thereto.

The membrane 40 may have a thickness from 3 to 5 μm, and may be formed of a material that is biocompatible, dysoxidative, noncorrosive, and electric resistive. In detail, the membrane 40 may be prepared by using a photosensitive material that is easily fabricated, such as Durimide, via a photolithography process, but is not limited thereto. According to an embodiment of the present invention, a method of preparing the membrane 40 may include: washing a silicon wafer to prepare it as a substrate; disposing an aluminum thin film having a thickness from 100 to 200 nm as a sacrifice film on the silicon wafer; stacking a Durimide film having a thickness from 5 to 7 μm on the aluminum thin film; forming a plurality of pores 41 on the Durimide film via a photolithography process; obtaining the membrane 40 having a thickness from 3 to 5 μm by processing the stacked silicon wafer from 30 to 60 min at 300-400° C.; and obtaining the membrane 40 with the pores 41 by corroding the aluminum thin film (sacrifice film) in a low concentration hydrochloric solution. However, the method is not limited thereto, and it is obvious to one of ordinary skill in the art that the membrane 40 may be prepared using any method.

According to an embodiment of the present invention, the first and second chambers 10 and 20 may be formed of a material that is biocompatible, dysoxidative, noncorrosive, and electric resistive. In detail, polymethylmethacrylate (PMMA) or polydimethylsiloxane (PDMS) may be used as the material, but the material is not limited thereto.

FIG. 4 is a perspective view of the first or second chamber 10 or 20 according to an embodiment of the present invention. A structure of the membrane 40 blocking one surface of the first or second chamber 10 or 20 is not shown in FIG. 4.

A cross-sectional area (depth×width) of a space formed inside the first or second chamber 10 or 20 may be wider than an area where the pores 41 are distributed in the membrane 40, and in detail, the depth of the space may be from 80 to 200 μm or from 80 to 150 μm.

A first electrode 11 is combined to the first chamber 10, and a second electrode 21 is combined to the second chamber 20. Here, the first and second electrodes 11 and 21 may be disposed in parallel to the membrane 40 that divides the first and second chambers 10 and 20. Referring to FIG. 4, an insert unit 12 and 22, where the first and second electrodes 11 and 21 are inserted and fixed on a surface of the first and second chambers 10 and 20 facing the membrane 40, may be formed so as to fix the first and second electrodes 11 and 21.

The first and second electrodes 11 and 21 may have a plate shape, and may be formed of a material that is biocompatible, dysoxidative, noncorrosive, and electric conductive. Examples of such a material include gold, platinum, and titanium, but are not limited thereto.

According to an embodiment of the present invention, the microdevice may further include an upper cover 30 combined to tops of the first and second chambers 10 and 20.

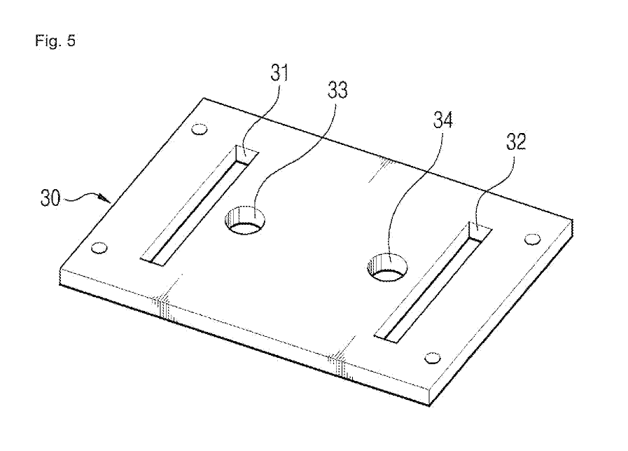

FIG. 5 is a perspective view of the upper cover 30 according to an embodiment of the present invention.

According to an embodiment of the present invention, the upper cover 30 may include a first inlet hole 33 for injecting a sample into the first chamber 10, and a second inlet hole 34 for injecting a sample into the second chamber 20.

According to another embodiment of the present invention, the upper cover 30 may include a first electrode insert hole 31 and a second electrode insert hole 32, and the first and second electrodes 11 and 21 may receive a predetermined voltage from the power supply unit by being exposed to the outside the microdevice respectively through the first and second electrode insert holes 31 and 32.

According to another embodiment of the present invention, the first electrode insert hole 31 and the second electrode insert hole 32 may be respectively formed farther outside than the first inlet hole 33 and the second inlet hole 34 so that the first and second more easily exist between the first and second electrodes 11 and 21

According to an embodiment of the present invention, the upper cover 30 may have a thickness from 2 to 5 mm, and may be formed of a material that is biocompatible, dysoxidative, noncorrosive, and electric resistive. In detail, polymethylmethacrylate (PMMA) or polydimethylsiloxane (PDMS) may be used as the material, but the material is not limited thereto.

At least one hole may be formed through the membrane 40, the first chamber 10, the second chamber 20, and the upper cover 30 at the same corresponding locations, and at least one bolt or the like may be inserted through the at least one hole, thereby preparing the microdevice in which each of structures are stacked on each other. The at least one hole may be formed at four corners of each structure, but is not limited thereto.

An embodiment of the present invention provides a method of fusing cells, the method including: providing the microdevice; injecting the first cell into the first chamber and injecting the second cell into the second chamber; applying an alternating current (AC) voltage between the first and second electrodes such that the first and second cells are disposed across the pore of the membrane according to a positive dielectrophoresis; performing electroporation by applying direct current (DC) pulses between the first and second electrodes; applying an quasi-damping AC voltage between the first and second electrodes such that the electroporated first and second cells are fused by being adjacently disposed to each other according to a positive dielectrophoresis; and obtaining the fused hybrid cells.

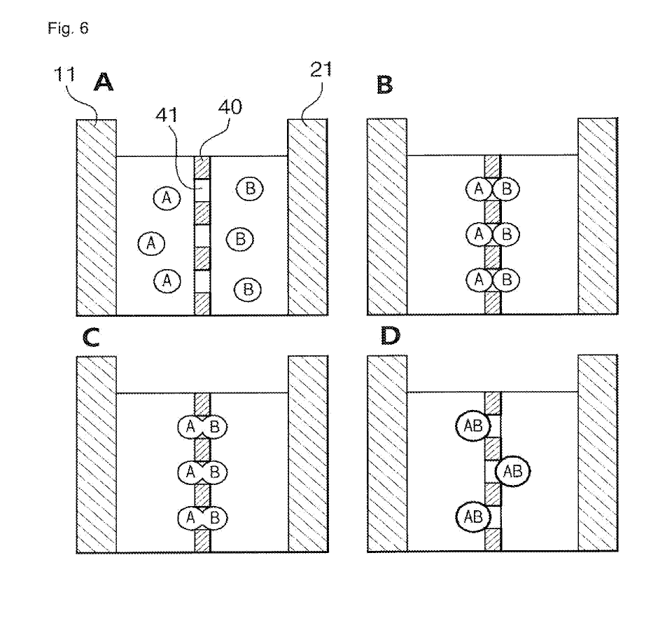

FIGS. 6A through 6D are schematic internal cross-sectional views for describing operations of a microdevice for fusing cells. The operations will now be described with reference to FIGS. 6A through 6D.

When a first cell is injected into a first chamber and a second cell is injected into a second chamber of the microdevice, the first and second cells do not pass through the membrane 40 and separately exist respectively in the first and second chambers as shown in FIG. 6A.

Then, an AC voltage (amplitude: 2-20V, frequency: 0.2-3 MHz) is applied between the first and second electrodes 11 and 21 such that the first and second cells are arranged across the pores 41 of the membrane 40 according to positive dielectrophoresis. The diameters of the pores 41 of the membrane 40 are formed to be smaller than the smallest diameter among the first and second cells and an electric field is most strongly formed in such a micro-pore. Accordingly, the first and second cells are adjacently arranged without passing through the pores 41 of the membrane 40 according to positive dielectrophoresis, as shown in FIG. 6B. Then, electroporation is performed on the first and second cells that are adjacently arranged by applying DC pulses (amplitude: 6-50V, duration: 10-500 μs, interval of two pulses: 0.1-10 s, pulses: 1-100) between the first and second electrodes 11 and 21. When the DC pulses are applied, the first and second cells are reversibly electroporated as shown in FIG. 6C.

Next, an quasi-damping AC voltage (amplitude: 1-10 V, frequency: 0.2-3 MHz, attenuation rate: −0-90%/min) is applied between the first and second electrodes 11 and 21 such that the electroporated first and second cells are adjacently disposed and fused according to positive dielectrophoresis, as shown in FIG. 6D. The first and second cells are fused together by passing through the pores 41 of the membrane 40, and the fused first and second cells exist in the first or second chambers.

Then, a solution having a high dielectric constant is injected through an inlet hole, and an AC voltage (amplitude: 1-10V, frequency: 0.2-3 MHz) is applied between the first and second electrodes 11 and 21, thereby obtaining the fused first and second cells through an outlet hole according to negative dielectrophoresis. A PBS buffer solution may be used as the solution having a high dielectric constant, and the fused first and second cells may be obtained by using a syringe pump or electrophoresis, but are not limited thereto.

According to the present invention, the first and second cells across the membrane may be arranged in an one-to-one manner between the first and second electrodes, and thus the first and second cells having different traits can be smoothly fused in an one-to-one manner when an electric signal are sequentially applied thereto.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A microdevice for fusing cells comprising:
a membrane with a plurality of pores having a diameter smaller than the smallest diameter among the first and second kinds of cells;
a first chamber where the first cell is located and a second chamber where the second cell is located, wherein the membrane is disposed therebetween;
a first electrode combined to the first chamber;
a second electrode combined to the second chamber; and
a power generator applying a voltage to the first and second electrodes.

2. The microdevice of claim 1, further comprising an upper cover combined to the tops of the first and second chambers.

3. The microdevice of claim 2, wherein the upper cover comprises a first inlet hole for injecting a sample into the first chamber, and a second inlet hole for injecting a sample into the second chamber.

4. The microdevice of claim 2, wherein the upper cover comprises a first electrode insert hole and a second electrode insert hole, and the first and second electrodes are exposed to the outside of the microdevice respectively through the first electrode insert hole and the second electrode insert hole to receive the voltage from the power generator.

5. The microdevice of claim 4, wherein the first electrode insert hole and the second electrode insert hole are formed farther outside than the first inlet hole and the second inlet hole.

6. The microdevice of claim 1, wherein the first and second electrodes are disposed parallel to the membrane.

7. The microdevice of claim 1, wherein a thickness of the membrane is from 3 to 5 μm.

8. The microdevice of claim 1, wherein the diameter of the plurality of pores of the membrane is smaller than the smallest diameter among the two kinds of cells by 1 to 15 μm.

9. A method of fusing cells, the method comprising:
providing the microdevice of claim 1;
injecting the first cell into the first chamber and injecting the second cell into the second chamber;
applying an alternating current (AC) voltage between the first and second electrodes such that the first and second cells are disposed across the pore of the membrane according to a positive dielectrophoresis;
performing electroporation by applying direct current (DC) pulses between the first and second electrodes;
applying an quasi-damping AC voltage between the first and second electrodes such that the electroporated first and second cells are fused by being adjacently disposed to each other according to a positive dielectrophoresis; and
obtaining the fused hybrid cells.

10. The microdevice of claim 3, wherein the upper cover comprises a first electrode insert hole and a second electrode insert hole, and the first and second electrodes are exposed to the outside of the microdevice respectively through the first electrode insert hole and the second electrode insert hole to receive the voltage from the power generator.
